(19) United States
(12) Patent Application Publication
(10) Pub. No.: US 2026/0320159 A1
(43) Pub. Date: Oct. 15, 2026

(54) SELF-EXPLAINABLE GRAPH MACHINE LEARNING APPROACH FOR NODE CLASSIFICATION

(71) Applicant: NEC Laboratories Europe GmbH, Heidelberg (DE)

(72) Inventors: Federico ERRICA, Heidelberg (DE); Zhao XU, Heidelberg (DE)

(21) Appl. No.: 19/104,255

(22) PCT Filed: Sep. 27, 2023
(86) PCT No.: PCT/IB2023/059596

§ 371 Date: Feb. 17, 2025

(30) Foreign Application Priority Data
Feb. 3, 2023 (EP) 23154902

(51) Int. Cl. G06F 18/2451; G06F 18/22; G06N 3/042; G06N 3/0455; G16H 50/20

(52) U.S. Cl. CPC G06F 18/2451; G06F 18/22; G06N 3/042; G06N 3/0455; G16H 50/20

(57) ABSTRACT

A computer-implemented method provides self-explainable machine learning node classification for a machine learning model that encodes a graph in latent space. The graph has nodes and edges semantically connecting pairs of the nodes in an original input space. The method includes: embedding learned prototypical nodes in the latent space of the model; predicting a class from for a particular node using the model; determining similarity scores between a latent representation of the particular node in the latent space and each of the learned prototypical nodes; mapping, into the original input space, the latent representation of the nodes and the learned prototypical nodes; and providing an explanation of the predicted class for the particular node based on the mapped prototypical nodes. The present invention can be used in a variety of applications including, but not limited to, several anticipated use cases in drug development, medical diagnostics/applications and in healthcare.

### CROSS REFERENCE TO RELATED APPLICATIONS

This application is a U.S. National Phase application under 35 U.S.C. § 371 of International Application No. PCT/IB2023/059596, filed on Sep. 27, 2023, and claims benefit to European Patent Application No. EP 23154902.3, filed on Feb. 3, 2023, the entire disclosure of which is hereby incorporated by reference herein. The International Application was published in English on Aug. 8, 2024 as WO 2024/161197 A1 under PCT Article 21(2).

### FIELD

The present disclosure relates to a method, system, and computer-readable medium for building prototypes for self-explainable graph machine-learning approach for node classification.

### BACKGROUND

Artificial Intelligence (AI) has emerged as a transformative technology with an ever-expanding role in making accurate predictions across various domains. AI's usefulness lies in its unparalleled ability to process vast amounts of data, uncover hidden patterns, and extrapolate insights, enabling business and industries to make informed decisions. From weather forecasting to healthcare diagnoses, AI has proved to be a powerful tool for enhancing the accuracy and reliability of predictions. The integration of machine learning algorithms allows AI systems to continuously improve their predictive capabilities as they learn from new data, making them adaptive and efficient in keeping up with dynamic environments. As a result AI-powered prediction models have become indispensable assets, driving innovation, optimizing processes, and empowering individuals and organizations to proactively respond to future scenarios.

However, there remains an issue with assessing the trustworthiness of an AI system's predictions. To this end, an emerging requirement for creating a trustworthy AI system is the ability to provide explainable predictions. The concept here is to give a human an explanation in order to garner trust in a machine learning model's prediction, yet the rationale must be human understandable. Human-understandability, however, is difficult to achieve, particularly when the input data is structured as a graph of interconnected entities and the individual entities need to be classified.

Moreover, in data-sensitive domains like healthcare, an additional requirement should be met: an explanation cannot contain sensitive-data related to other patients. This is a very challenging requirement for current graph AI methods, which usually provide an explanation for a node classification in terms of other nodes and connections in the graph. In principle, this can cause a serious privacy issue, including violating the European Union's General Data Protection Regulation (GDPR). Thus, finding privacy-law compliant (e.g., GDPR-compliant) alternatives is of paramount importance.

### SUMMARY

According to an aspect of the present disclosure, a computer-implemented method is provided for self-explainable machine learning node classification for a machine learning model that encodes a graph in latent space. The graph has a set of nodes and a set of edges semantically connecting pairs of the nodes in an original input space. The method includes: embedding learned prototypical nodes in the latent space of the model; predicting a class from a set of classes for at least a particular node of the nodes using the model; determining similarity scores between a latent representation of the particular node in the latent space and each of the learned prototypical nodes; mapping, into the original input space, the latent representation of the nodes and the learned prototypical nodes; and providing an explanation of the predicted class for at least the particular node based on the mapped prototypical nodes in the original input space. The method has applications including, but not limited to digital medicine and personalized healthcare, for example, for AI-assisted drug development (AIDD) or patient predictions, public safety, smartcities and other machine learning applications using graphs.

The present invention can be used in a variety of applications including, but not limited to, several anticipated use cases in drug development, medical diagnostics/applications and in healthcare.

### BRIEF DESCRIPTION OF THE DRAWINGS

Embodiments of the present disclosure will be described in even greater detail below based on the exemplary figures. The present disclosure is not limited to the exemplary embodiments. All features described and/or illustrated herein can be used alone or combined in different combinations in embodiments of the present disclosure. The features and advantages of various embodiments of the present disclosure will become apparent by reading the following detailed description with reference to the attached drawings which illustrate the following:

FIG. 1 illustrates an exemplary embodiment of a system and method implemented according to aspects of the present disclosure; and

FIG. 2 is a block diagram of an exemplary processing system, which can be configured to perform any and all operations disclosed herein.

### DETAILED DESCRIPTION

Aspects of the present disclosure provide a self-explainable graph machine learning approach for node classification tasks that can also generate human-understandable predictions via prototypes associated with a target class. The disclosed machine-learning approach iteratively learns from human inputs and updates its prototypes and explanations accordingly.

Compared to the present state of the art, the methodology provided in the present disclosure assumes that humans are not good at interpreting subgraphs as explanations for node prediction tasks, and instead focuses on generating a new, connected node that represents the “ideal candidate” for a specific output class. In addition, aspects of the present disclosure provide an easy mechanism to incorporate human-feedback in the loop and improve the explanations, which cannot be adopted by explainable graph artificial intelligence (AI) methods that do not adopt an auto-encoder architecture. By simultaneously predicting the class and decoding the prototypical representation in the original graph space, embodiments implemented according to the present disclosure can increase the trust level of users. Such users can also ask for additional explanations by generating variants of the prototypical representation from the associated distribution; in contrast, methods in the literature generally produce a fixed amount of possible explanations.

Embodiments implemented according to the present disclosure provide technical improvements in producing human-understandable predictions in a node-classification scenario. For example, embodiments implemented according to the present disclosure provide at least the following functionalities (the “desiderata”), which are generally not available in existing systems:

Easy-to-understand explanations: most existing methods return subgraphs that are merely considered useful for the predicted node/graph class. See, e.g., Zhang, et al., “ProtGNN: Towards Self-Explaining Graph Neural Networks,” AAAI-22, 9127-9135 (2022) (available at <<https://ojs.aaai.org/index.php/AAAI/article/download/20898/20657>>); Shin et al., “PAGE: Prototype-Based Model-Level Explanations for Graph Neural Networks,” arXiv, 1-18 (2022) (available at <<https://arxiv.org/pdf/2210.17159.pdf>>); Dai and Wang, “Towards Prototype-Based Self-Explainable Graph Neural Network,” Assn. for Computing Machinery Conference 2017, 1-9 (2017) (available at <<https://arxiv.org/pdf/2210.01974.pdf>>); Magister, et al., “Encoding Concepts in Graph Neural Networks,” arXiv, 1-24 (August 2022) (available at <<https://arxiv.org/pdf/2207.13586.pdf>>) (the entire contents of each of which are hereby incorporated by reference herein). In a general context, humans without precise domain knowledge cannot understand the meaning of the subgraph, and are therefore confused. In contrast, embodiments of the present disclosure provide human-understandable explanations of the graph.

Model-level explanations: most existing methods rely on post-hoc explanations, that is, the explanations are extracted after the model is trained. See, e.g., Huang, et al., “GraphLIME: Local Interpretable Model Explanations for Graph Neural Networks,” IEEE Transactions on Knowledge and Data Engineering, 35:7, 6968-6972, (June 2022) (available at <<https://ieeexplore.ieee.org/stamp/stamp.jsp?tp=&arnumber=9811416>>); Ying et al., “GNNExplainer: Generating Explanations for Graph Neural Networks,” 33rd Conference on Neural Information Processing Systems, 1-13 (2019) (available at <<https://arxiv.org/pdf/1903.03894.pdf>>) (the entire contents of each of which are hereby incorporated by reference herein). Embodiments of the present disclosure, on the other hand, learn to generate explanations, together with decisions, during training.

Generating as many explanations as requested: while there exist methods that provide a set of explanations for a given prediction, additional explanations cannot usually be provided. On the one hand, the generation of too many explanations can confuse the human; on the other hand, having too few explanations cannot be enough for the human to trust a predictive system. Embodiments of the present disclosure, however, can address a scenario where a human may not immediately understand a given explanation by proposing a new, but still valid, alternative reason why a certain prediction was made.

Embodiments implemented according to the present discourse are enabled, at least in part, to provide at least aforementioned functionalities by implementing one or more of the following technical improvements:

Providing an explanation as a learned prototypical node in the graph: embodiments of the present disclosure learn “template/prototypical nodes” that are representative for a specific class. They offer humans a simple interpretation in the original space of features: if node features are of easy human comprehension, so will be the prototypical node.

Prototypical nodes live in the embedding space: embodiments of the present disclosure train a model to produce the aforementioned prototypes, while also classifying nodes. A prototype is embedded in the latent space of the model, and it can then be decoded as a newly generated node of the original graph. This way, post-hoc explanations are avoided, and the explanation is functional to the prediction of a particular class.

Prototypical nodes are represented as parameters of distributions: in embodiments of the present disclosure, a prototypical node is encoded in latent space as the parameters of some distribution, e.g., Gaussian. This way, one can consider the most likely prototypical node or generate less “probable” explanations by sampling from this specific Gaussian. Hence, there is no limit to the number of explanations that a user can ask for.

The above collection of three characteristics are not present in existing systems. Embodiments of the present disclosure implement these characteristics via a variational graph auto-encoder (VGAE) in a latent variable modeling framework. The VGAE is a machine learning model trained to find a lower dimensional yet meaningful representation of the nodes. Differently from what has been done in the past, this VGAE is composed of a generative distribution defined as a mixture of K distributions. The variational distribution, which tries to approximate the true posterior of the data, is instead implemented in a more classical way.

Embodiments of the present disclosure also utilize unstructured (i.e., vectorial) input data, which relies on the notion of prototypes, or prototypical representations, which are abstract concepts carrying useful information, while not being identifiable with specific entities (e.g., patients).

According to a first aspect of the present disclosure, a method is provided that implements a self-explainable graph machine-learning scheme for node classification. The method learns prototypical representations in latent space to interpret the predictions of a graph's entities by means of a variational graph auto-encoder. The prototypical representations can be mapped from latent space back to the original graph space by means of a learned decoder to be easily inspected by the users. The method can generate a potentially infinite number of prototypical representations to increase the trust level of the user by sampling from the distributions associated with the latent prototypes. The method can provide human-understandable evidence that the model has learned to associate a specific prototypical representation with a given output class, by inspecting the weights of the interpretable classifier built upon the prototypes and the similarity of the input with respect to them.

According to a second aspect of the present disclosure, a method is provided for prototype-based, self-explainable node classification. The method includes:
1) recording or collecting information (e.g., from a patient network) and pre-processing the information into a graph with node features;
2) embedding entities in the latent space learned by the method;
3) making interpretable predictions about the individual entities by comparing them with the learned prototypical representations; and
4) providing an explanation for the predictions in terms of prototypical representations decoded as nodes connected to the original graph.

In an embodiment of one of the aspects, the method may additionally include iteratively updating the explanation based on some domain-related input.

According to a third aspect of the present disclosure, a computer-implemented method is provided for self-explainable machine learning node classification for a machine learning model that encodes a graph in latent space. The graph has a set of nodes and a set of edges semantically connecting pairs of the nodes in an original input space. The method includes: embedding learned prototypical nodes in the latent space of the model; predicting a class from a set of classes for at least a particular node of the nodes using the model; determining similarity scores between a latent representation of the particular node in the latent space and each of the learned prototypical nodes; mapping, into the original input space, the latent representation of the nodes and the learned prototypical nodes; and providing an explanation of the predicted class for at least the particular node based on the mapped prototypical nodes in the original input space.

In an embodiment of the third aspect, the method may further include: recording, collecting, or obtaining information from a target domain and processing the information into the graph in the original input space.

In an embodiment of the third aspect, the embedding of the learned prototypical nodes in the latent space of the model includes: generating K distributions in the latent space; and determining each of K means respectively from one of the K distributions, each of the K means being one of the learned prototypical nodes. The method may further include encoding the graph in the latent space, the encoding of the graph including: for each respective node of the nodes in the graph in the original input space: producing a respective distribution in the latent space, a mean of the respective distribution being a respective latent representation of the respective node; and determining a probability that the respective latent representation of the node is generated by one of the K distributions.

In an embodiment of the third aspect, K may be a predetermined integer provided by a user, and each of the K distributions are continuous distributions.

In an embodiment of the third aspect, an encoder executes the encoding of the graph in latent space, the encoder having a graph convolutional network or a graph attention network.

In an embodiment of the third aspect, predicting the class of the particular node includes: obtaining a similarity vector of K entries from the similarity scores; and processing the similarity vector, by a linear classifier, to predict the class for the particular node.

In an embodiment of the third aspect, the method further includes: inspecting weights of the linear classifier, the relative weights providing a relative indication of how important being similar to a certain one of the learned prototypical nodes is for predicting the classes.

In an embodiment of the third aspect, the linear classifier includes an objective function with a regularization term to favor sparse similarity vectors.

In an embodiment of the third aspect, a trained decoder performs the mapping, into the original input space, the latent representation of the nodes and the learned prototypical nodes. The decoder may also include a graph machine learning model having been trained on the basis that a loss trains the decoder to reconstruct the original node feature from the latent representation.

In an embodiment of the third aspect, the graph machine learning model is further configured to reconstruct the original edges between the nodes using a trained graph auto-encoder.

In an embodiment of the third aspect, the method may further include: providing an alternative explanation for the prediction of the class by: selecting a distribution, from among the K distributions, determined to be relevant for the class; sampling, from the selected distribution, a vector in the latent space; mapping, into the original space, the sampled vector; and providing the alternative explanation based on the sampled vector mapped into the original space.

In an embodiment of the third aspect, a decoder may be provided and includes a trained machine learning model that executes the mapping of the prototypical nodes into the original input space. The method may further include: receiving an input from a user proposing a modification to a feature of a selected one of the prototypical nodes mapped into the original input space; and re-training only the decoder based on the input such that the selected prototypical node is decoded as indicated by the user.

In an embodiment of the third aspect, the target domain is a patient network with the information of the target domain being partially available data of patients comprising vital measurements and laboratory exam results, the learned prototypical nodes represent learned prototypical patients associated with a clinical diagnosis, and the set of classes is a set of diseases.

As a person of ordinary skill in the art would appreciate, the embodiments of the third aspect may be variously combined all within the scope of the present disclosure.

According to a fourth aspect of the present disclosure, a computer system is provided that includes one or more hardware processors which, alone or in combination, are configured to provide for the execution of the method of each of the above-described aspects, including the embodiments as variously implemented or combined.

According to a fifth aspect of the present disclosure, a tangible, non-transitory computer-readable medium is provided, which has instructions thereon which, upon being executed by one or more hardware processors, alone or in combination, provide for execution of the method of each of the above-described aspects, including the embodiments as variously implemented or combined.

The above-discussed and additional features and advantages of various embodiments of the present disclosure are apparent in view of the following exemplary embodiments described with reference to the drawings.

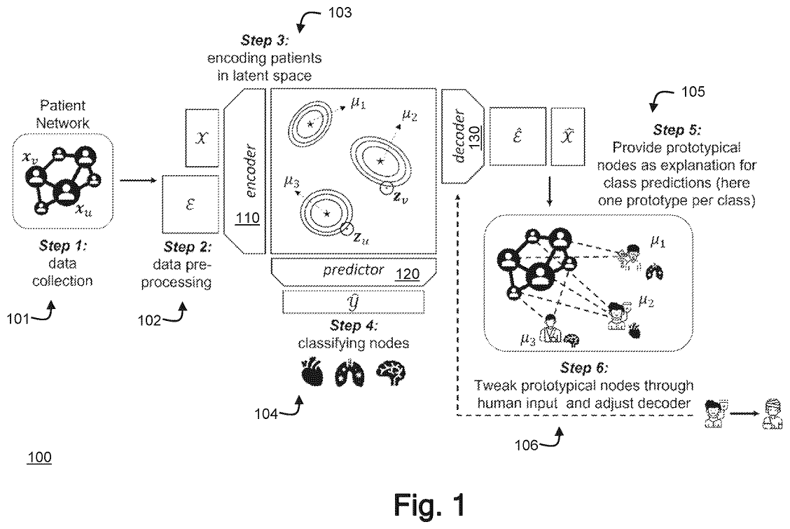

FIG. 1 illustrates an embodiment of a method and system 100 implemented according to the present disclosure. The method implements a variational formulation of the problem to address at least the above-listed desiderata, and deals with three different machine learning tasks to be optimized using backpropagation: 1) node classification; 2) node reconstruction; 3 and link prediction.

### Input and Problem Definition

As shown in FIG. 1, in a first step 101, an input set of data is collected or otherwise obtained. The input includes a collection of entities that are semantically connected to each other so that they can be represented as a graph. For example, the collected data may undergo, in a second step 102, a pre-processing operation to, inter alia, create a graph g of the collected data.

The graph g (sometimes referred to as a network) is a machine understandable data structure that provides a semantically-structured representation of information. The graph represents entities (e.g., patients, doctors, conditions, illnesses, vital measurements, etc.) as “nodes”, and relationships between entities (e.g., has condition, attended by, blood results, has weight, etc.) as “edges” in the graph. Essentially, two nodes connected by a relationship form a “fact” captured by the graph. The graph g may be characterized as a tuple (V, [symbol] , [symbol] , [symbol] ), where V is the set of nodes (e.g., patients), [symbol] is the set of directed edges connecting pairs of nodes, [symbol] is the domain of node features, and (optionally) [symbol] is the domain of edge features. As used herein, the domain of node features refers to the set of possible attributes, characteristics, or properties associated with individual nodes within the graph, and the domain of edge features refers to the set of possible attributes, characteristics or properties that can be attributed to the edges.

From the graph g, a feature vector can be derived. A feature vector is a structured-representation of attributes or characteristics associated with either a node or an edge. Thus, the feature vector encapsulates relevant information about a particular entity or edge of the graph g. Feature vectors can serve as input data for machine learning algorithms, enabling the algorithm to analyze and made prediction based on the provided attributes. In the present disclosure, xᵤ∈ [symbol] denotes the feature vector of a node u (e.g., containing patient measurements, such as heart-rate, blood information, weight, etc.).

The task (or problem) of the embodiment of FIG. 1 is to classify each node of the graph, while also providing explanations for those classifications in the form of prototypical nodes.

A “prototypical node” refers to a representative or characteristic node within the graph. A prototypical node embodies typical attributes or features that are common among a subset of nodes the graph. The prototypical node exists in the original space of the graph, and it also has corresponding representation in a latent space, latent space being a n-dimensional Euclidean space where the nodes of the original space of the graph are mapped to, with n typically (but not necessarily) being a small number.

### The Generative Distribution of Prototypical Nodes

Below, the (learned) generative distribution of the variational graph auto-encoder (VGAE) method implemented to the present disclosure is described.

The generative distribution of the VGAE method is defined as a mixture of K distributions in a latent space of dimension d. For instance, if a user chooses K=3 and a Gaussian distribution, then the means of the individual Gaussians μ₁, μ₂, μ₃∈ [symbol] ^{d} will represent the main prototypical representations in latent space (as shown in FIG. 1). In other words, the “main prototypical representations” are those associated with the mean of a Gaussian—while any value close to the mean can be considered a prototypical representation, the “main” representation associated with the mean is statistically more likely to be representative. To interpret how important each prototypical representation is for a given class, a classification model is used (see below). Also, these Gaussians may be sampled from to get alternative plausible representations that are associated with a given class (further discussed below). While the present disclosure is discussed in terms of Gaussian distributions, aspects of the present disclosure may be implemented using any (or a mixture of) distributions with continuous support that is differentiable, e.g., the Poisson distribution.

### Encoding Nodes in Latent Space

Returning to FIG. 1, in a third step 103, an encoder 110 implements a variational distribution according to the VGAE framework provided in the present disclosure. Namely, for each node u, the encoder 110 produces a distribution with mean zᵤ and the probability that that node belongs to one of the K components of the mixture of distributions. The encoder 110 can be variously implemented using graph machine learning methods, such as with a graph-convolutional network (GCN) and a graph attention network (GAT). However, the present disclosure is not limited to a specific machine learning model, but rather may be implemented with a machine learning model configured for graph structured data that converts nodes of an input graph in to points in latent space.

### Interpretable Predictions for Each Node

To carry out an interpretable prediction for a node u, the concept of “prototype matching” is used. Using probabilistic techniques (e.g., using Bayes' theorem), a similarity score between zᵤ and each of the prototypical representations μ¹, μ², μ³ are computed. More specifically, the probability that a node has been “generated” by one of the K=3 distributions (e.g., Gaussians) is computed. Therefore, a similarity vector of K entries is obtained, which is feed into a linear classifier (e.g., a logistic regressor) to produce a class yᵤ for the specific node u. As shown in FIG. 1, a predictor 120 carries out the fourth step 104 carries out the above functionality to classify the nodes.

An insight of the present disclosure is that the weights of the linear model can be inspected to determine how important being similar to a certain prototype is for predicting a certain class. To further improve understandability of the predictions, a regularization term can be added to the objective function of the classifier, which favors sparse similarity vectors. The objective function can be implemented, for example, using the evidence lower bound function (ELBO), which is well understood in the art. The regularization term is one the minimizes the entropy of the variational distribution computing the cluster assignment probability of each node in the input graph.

The linear classifier will predict a probability score for each class given an input representation of a prototype μ^{x}. If the score for a given class is sufficiently high (e.g., satisfies a threshold imposed by the user), the system determines that the prototype is “particularly important” for that specific class.

### Decoding of the Original Graph

In a fifth step 105, a learned decoder 130 maps the latent representations of each node zᵤ back to xᵤ. The decoder 130 can be implemented, for example, with a multi-layer perceptron (MLP) or another graph machine learning model. A loss trains the decoder to reconstruct the original node feature from the latent representation. In addition, the decoder 130 may include a link prediction decoder, such as graph auto-encoder (GAE), in order to reconstruct the original links between the nodes. Here, to train the GAE, a binary classification loss can be used in conjunction with a negative sampling of non-existing links.

The decoder may be implemented using one of many well-understood in the art machine learning models configured for vectorial data, which convert latent representations of the nodes of an input graph into node features in the original graph space. The machine learning model implemented for the decoder additionally reconstructs the connections between the nodes.

### Explaining Predictions Through Prototypes

Also in the fifth step 105, the decoder 130 maps the prototypical representations μ¹, μ², μ³ to the original input space of nodes. This allows the system to interpret the meaning of each learned prototypical representation in a format that a human might comprehend.

In addition, it is possible to connect such new nodes (i.e., the re-mapped node representations) to the most similar learned nodes (i.e., the prototypical nodes) by using the link prediction decoder for visualization purposes. If it has been determined that a learned prototype is particularly important for a particular class (as determined above in the fourth step 104), the user may be told that a “prototypical” representative for a specific class has been identified, and its feature values, e.g., a “proto-patient”, can be shown with certain properties (e.g., determined by the decoder) that most likely lead to its classification (e.g., cardiovascular issues). This, together with the similarity vector of a specific node, helps the human understand what the model has learned.

In addition, the users might ask for different explanations that are representative for a certain class c. If that is the case, the system picks some of the distributions deemed relevant (e.g., those passing the threshold of “particularly important”) for the class c from the generative model, samples a value/vector in latent space from one or all of them and decodes it as nodes in the original graph space as done before.

### Improving Prototypical Representations Through New Inputs

Upon inspection of the learned prototypical representations, the system allows users to provide input by proposing modifications to some features of a prototypical representation (e.g., the input may be indicating that a value for a feature predicted by a decoder should be higher/lower for a specific prototypical representation). In that case, the system is imbued with this input by re-training only the node features decoder 130, while making sure that that specific prototypical latent representation is now decoded as expected. This is represented as the sixth step 106 in FIG. 1. Changing the decoder does not alter the latent representation of nodes and, therefore, leaves the classification performances unchanged while improving the explanation quality.

Embodiments of the present invention thus provide for general improvements to computers in machine learning systems by providing the self-explainable graph machine learning approach. Moreover, embodiments of the present invention can be practically applied to use cases to effect further improvements in a number of technical fields that use or could benefit from graph-based machine learning including, but not limited to, medical (e.g., digital medicine, personalized healthcare, AI-assisted drug or vaccine development (AIDD or Oncoimmunity), etc.), material development, public safety and smart cities (e.g., automated traffic or vehicle control, smart districts, smart buildings, smart industrial plants, smart agriculture, energy management, etc.).

### Exemplary Implementation 1: Explaining Predictions of Clinical Risk in a Patient Network

Use Case: Predicting clinical risk in hospitals using AI can accelerate diagnoses of patients that could be subject to determined illnesses (e.g., sepsis, acute kidney injuries, etc.). However, doctors might not be willing to blindly trust AI predictions without some intuitive knowledge/explanation of how the predictive process works. An implementation of a system and method according to the present disclosure can be used to maintain the same predictive accuracy as other methods, while also providing an intuitive explanation (through the prototypical representations) of why a certain patient has been classified that way. The prediction can be fed into a predictive system that outputs, together with a prediction of the clinical risk, a set of additional exams to take, e.g., by providing the configuration of a complete blood count (CBC) blood test machine. In addition, the doctor can provide feedback on the prototypical representations that can be easily integrated into the system.

Data Source: A patient network, which consists of partially available data of patients. This data includes, but it is not limited to, basic vital measurements and laboratory exams, such as heart rate, oxygen saturation, weight, height, glucose, temperature, pH, etc. A blood sample of the patient is available for further use.

The Approach: The system implemented according to the present disclosure will predict the clinical risk of each patient through his/her association with a learned prototypical patient. This association is presented to the doctor as a form of explanation, e.g., the prototypical patient is a good representative of patients with heart diseases and the current patient is “close” to it in latent space. This explanation provides insights into why a certain decision was made by the system, and increases the level of trust of the doctors. In addition, the doctor can gain further insights by asking for more prototypical representations to be generated from the main one, which are meant to further convince the doctor that the proposed system has learned meaningful associations between patient features and clinical risk.

Output: Prediction of clinical risk and information on one or more prototypical patients that are representative for that specific clinical risk as a form of explanation.

### Exemplary Implementation 2: Patient Stratification: Predicting the Response to Treatment in Clinical Trials and Identifying the Ideal Candidates

Use Case: One of the factors which mostly impact clinical trials' budget, and ultimately their outcome, is the patient selection process. Many times, patients who should not be eligible for the treatment (because the trial will not cause a positive response) are enrolled anyway, and this can cause a huge monetary loss that ultimately leads to the failure of the trial itself. The rationale is to perform a stratification of patients using information at a genetic level, to predict earlier their response: this will result in a stratification between responders and non-responders. Given a network of patients, who are connected according to the distribution of their cell types, and partial knowledge about their response to a specific drug, the goal is to predict the response for the other patients, while also identifying an “ideal candidate” for it. Here, the assumptions include that patients with similar distributions of cell types react similarly to the drug and that the set of gene-cell information correlates with such distribution.

Data Source: A network of patients with genomic activity information (e.g., single cell RNA-sequencing), their response to a specific drug, and publicly available ontologies that can be used to build the graph.

The Approach: The system implemented according to the present disclosure will predict the patients' response to the drug according to his/her similarity to a learned “ideal candidate”. The method will produce ideal responders and non-responders, and embeds them in the patient graph, which can be used by the user (trial manager) to validate the results.

Output: Prediction of the patient's response to the drug (low/medium/high).

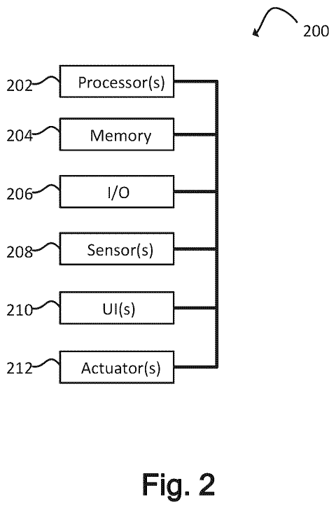

FIG. 2 illustrates a processing system 200 which can be representative of each of the computing systems disclosed herein. The processing system 200 can include one or more processors 202, memory 204, one or more input/output devices 206, one or more sensors 208, one or more user interfaces 210, and one or more actuators 212.

Processors 202 can include one or more distinct processors, each having one or more cores. Each of the distinct processors can have the same or different structure. Processors 202 can include one or more central processing units (CPUs), one or more graphics processing units (GPUs), circuitry (e.g., application specific integrated circuits (ASICs)), digital signal processors (DSPs), and the like. Processors 202 can be mounted to a common substrate or to multiple different substrates.

Processors 202 are configured to perform a certain function, method, or operation (e.g., are configured to provide for performance of a function, method, or operation) at least when one of the one or more of the distinct processors is capable of performing operations embodying the function, method, or operation. Processors 202 can perform operations embodying the function, method, or operation by, for example, executing code (e.g., interpreting scripts) stored on memory 204 and/or trafficking data through one or more ASICs. Processors 202, and thus processing system 200, can be configured to perform, automatically, any and all functions, methods, and operations disclosed herein. Therefore, processing system 200 can be configured to implement any of (e.g., all of) the protocols, devices, mechanisms, systems, and methods described herein.

For example, when the present disclosure states that a method or device performs task “X” (or that task “X” is performed), such a statement should be understood to disclose that processing system 200 can be configured to perform task “X”. Processing system 200 is configured to perform a function, method, or operation at least when processors 202 are configured to do the same.

Memory 204 can include volatile memory, non-volatile memory, and any other medium capable of storing data. Each of the volatile memory, non-volatile memory, and any other type of memory can include multiple different memory devices, located at multiple distinct locations and each having a different structure. Memory 204 can include remotely hosted (e.g., cloud) storage.

Examples of memory 204 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 204.

Input-output devices 206 can include any component for trafficking data such as ports, antennas (i.e., transceivers), printed conductive paths, and the like. Input-output devices 206 can enable wired communication via USB®, Display Port®, HDMI®, Ethernet, and the like. Input-output devices 206 can enable electronic, optical, magnetic, and holographic, communication with suitable memory 206. Input-output devices 206 can enable wireless communication via WiFi®, Bluetooth®, cellular (e.g., LTE®, CDMA®, GSM®, WiMax®, NFC®), GPS, and the like. Input-output devices 206 can include wired and/or wireless communication pathways. The input devices can be, especially, user devices such as smartphones, tablets, laptops or personal computers.

Sensors 208 can capture physical measurements of environment and report the same to processors 202, for example, cameras. User interface 210 can include displays, physical buttons, speakers, microphones, keyboards, and the like. Actuators 212 can enable processors 202 to control mechanical forces.

Processing system 200 can be distributed. For example, some components of processing system 200 can reside in a remote hosted network service (e.g., a cloud computing environment) while other components of processing system 200 can reside in a local computing system. Processing system 200 can have a modular design where certain modules include a plurality of the features/functions shown in FIG. 2. For example, I/O modules can include volatile memory and one or more processors. As another example, individual processor modules can include read-only-memory and/or local caches.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

Additional relevant background information on AI models can be found in, for example, Jiang et al., “Variational Deep Embedding: An Unsupervised and Generative Approach to Clustering,” IJCAI 2017, 1-22 (2017) (available at <<https://arxiv.org/pdf/1611.05148.pdf>>) (the entire contents of which is hereby incorporated by reference herein).

1. A computer-implemented method for self-explainable machine learning node classification for a machine learning model that encodes a graph in latent space, the graph comprising a set of nodes and a set of edges semantically connecting pairs of the nodes in an original input space, the method comprising:
embedding learned prototypical nodes in the latent space of the model;
predicting a class from a set of classes for at least a particular node of the nodes using the model;
determining similarity scores between a latent representation of the particular node in the latent space and each of the learned prototypical nodes;
mapping, into the original input space, the latent representation of the nodes and the learned prototypical nodes; and
providing an explanation of the predicted class for at least the particular node based on the mapped prototypical nodes in the original input space.

2. The method of claim 1, the method further comprising:
recording, collecting, or obtaining information from a target domain and processing the information into the graph in the original input space.

3. The method of claim 1,
wherein embedding the learned prototypical nodes in the latent space of the model comprises:
generating K distributions in the latent space; and
determining each of K means respectively from one of the K distributions, each of the K means being one of the learned prototypical nodes; and
the method further comprising encoding the graph in the latent space, the encoding of the graph comprising:
for each respective node of the nodes in the graph in the original input space:
producing a respective distribution in the latent space, a mean of the respective distribution being a respective latent representation of the respective node; and
determining a probability that the respective latent representation of the node is generated by one of the K distributions.

4. The method of claim 3,
wherein K is a predetermined integer provided by a user, and
wherein each of the K distributions are continuous distributions.

5. The method of claim 3, wherein an encoder executes the encoding of the graph in latent space, the encoder comprising a graph convolutional network or a graph attention network.

6. The method of claim 1, wherein predicting the class of the particular node comprises:
obtaining a similarity vector of K entries from the similarity scores; and
processing the similarity vector, by a linear classifier, to predict the class for the particular node.

7. The method of claim 6, the method further comprising:
inspecting weights of the linear classifier, the relative weights providing a relative indication of how important being similar to a certain one of the learned prototypical nodes is for predicting the classes.

8. The method of claim wherein the linear classifier comprises an objective function with a regularization term to favor sparse similarity vectors.

9. The method of claim 1, wherein a trained decoder performs the mapping, into the original input space, the latent representation of the nodes and the learned prototypical nodes, wherein the decoder comprises a graph machine learning model having been trained on the basis that a loss trains the decoder to reconstruct the original node feature from the latent representation.

10. The method of claim 9, wherein the graph machine learning model is further configured to reconstruct the original edges between the nodes using a trained graph auto-encoder.

11. The method of claim 1, the method further comprising:
providing an alternative explanation for the prediction of the class by:
selecting a distribution, from among the K distributions, determined to be relevant for the class;
sampling, from the selected distribution, a vector in the latent space;
mapping, into the original space, the sampled vector; and
providing the alternative explanation based on the sampled vector mapped into the original space.

12. The method of claim 1, wherein a decoder comprising a trained machine learning model executes the mapping of the prototypical nodes into the original input space, and wherein the method further comprises:
receiving an input from a user proposing a modification to a feature of a selected one of the prototypical nodes mapped into the original input space; and
re-training only the decoder based on the input such that the selected prototypical node is decoded as indicated by the user.

13. The method of claim 1, wherein the target domain is a patient network, with the information of the target domain being partially available data of patients, which comprises vital measurements and laboratory exam results, wherein the learned prototypical nodes represent learned prototypical patients associated with a clinical diagnosis, and wherein the set of classes is a set of diseases.

14. A computer system comprising one or more hardware processors which, alone or in combination, are configured to provide for the execution of a method for self-explainable machine learning node classification for a machine learning model that encodes a graph in latent space, the graph comprising a set of nodes and a set of edges semantically connecting pairs of the nodes in an original input space, the method comprising:
embedding learned prototypical nodes in the latent space of the model;
predicting a class from a set of classes for at least a particular node of the nodes using the model;
determining similarity scores between a latent representation of the particular node in the latent space and each of the learned prototypical nodes;
mapping, into the original input space, the latent representation of the nodes and the learned prototypical nodes; and
providing an explanation of the predicted class for at least the particular node based on the mapped prototypical nodes in the original input space.

15. A tangible, non-transitory computer-readable medium having instructions thereon which, upon being executed by one or more hardware processors, alone or in combination, provide for execution of a method for self-explainable machine learning node classification for a machine learning model that encodes a graph in latent space, the graph comprising a set of nodes and a set of edges semantically connecting pairs of the nodes in an original input space, the method comprising:
embedding learned prototypical nodes in the latent space of the model;
predicting a class from a set of classes for at least a particular node of the nodes using the model;
determining similarity scores between a latent representation of the particular node in the latent space and each of the learned prototypical nodes;
mapping, into the original input space, the latent representation of the nodes and the learned prototypical nodes; and
providing an explanation of the predicted class for at least the particular node based on the mapped prototypical nodes in the original input space.
